(19) United States
(12) Patent Application Publication
(10) Pub. No.: US 2002/0012947 A1
(43) Pub. Date: Jan. 31, 2002

(54) Biomarkers for the labeling, visual detection and quantification of biomolecules

(76) Inventors: Susan Ann Bevers, Waltham, MA (US); Rodrigo Bohn Andrade, Somerville, MA (US); Kiril Stefan Alexandrov, Cambridge, MA (US); Zoran Z. Zdraveski, Cambridge, MA (US)

Correspondence Address: HALE AND DORR, LLP, 60 STATE STREET, BOSTON, MA, 02109

(21) Appl. No.: 09/804,893

(22) Filed: Mar. 13, 2001

Related U.S. Application Data
(60) Provisional application No. 60/189,264, filed on Mar. 14, 2000.
(60) Provisional application No. 60/209,188, filed on Jun. 5, 2000.

(51) Int. Cl. G01N 33/53; C07H 21/04; C07K 14/00

(52) U.S. Cl. 435/7.1; 536/23.1; 530/350

(57) ABSTRACT

This invention relates to the detection of biomolecules. In particular, the invention relates to biomarkers for the labeling, visually detection and quantification of biomolecules. The invention provides visually detectable biomolecules and reagents for their preparation, as well as methods for visually detecting a biomolecule and for determining the size of a biomolecule. The labeled biomolecules of the invention are intensely colored and can be readily observed by visual inspection, without prior illumination or chemical or enzymatic activation.

### CROSS-REFERENCE TO RELATED APPLICATIONS

This application claims priority from U.S. Provisional Patent Application Ser. No. 60/189,264, filed on Mar. 14, 2000, and No. 60/209,188, filed on Jun. 5, 2000.

### BACKGROUND OF THE INVENTION

1. Field of the Invention

This invention relates to the detection of biomolecules. In particular, the invention relates to biomarkers for the labeling, visual detection and quantification of biomolecules. The invention provides visually detectable biomolecules and reagents for their preparation, as well as methods for visually detecting a biomolecule and for determining the size of a biomolecule.

2. Background of the Invention

Methods for detecting biomolecules typically rely upon the use of fluorescent, chemiluminescent, or radioactive biomarkers. Each of these classes of biomarkers suffers limitations, however. For example, the use of radioactive labels requires careful attention to safety and regulatory protocols, and disposal of the resultant radioactive waste is both costly and potentially hazardous.

Fluorescent dyes have been widely used as an alternative to radioactive biomarkers. However, background fluorescence from components present in a test sample may interfere with an accurate determination of the fluorescence of the fluorescent label. Furthermore, the label itself may be photolabile and exhibit bleaching when subjected to the fluorescence detection conditions.

Tizard R. et al., Proc. Natl. Acad. Sci. USA, 87: 4514-4518 (1990) describes the use of 1,2-dioxetanes as chemiluminescent probes. Upon chemical or enzymatic activation, the dioxetane decomposes, with the emission of light. These biomarkers suffer the disadvantage of being consumed when subjected to the detection protocol.

Perylenes and related dyes have high photochemical persistency (chemical, thermal, and photochemical stability) and high fluorescence quanta yield and are used in a variety of reprographic processes, solar cells, photovoltaic devices, and dye lasers. Bair, U.S. Pat. No. 4,719,236, teaches perylene derivatives useful as biocidal agents, particularly antitumor agents. However, perylene derivatives have been used primarily as pigments and fluorescent dyes. Müllen et al., U.S. Pat. No. 5,986,099 and U.S. Pat. No. 6,124,458, describe the synthesis of substituted quaterrylene tetracarboxylic acid diimides and their use as pigments or fluorescent dyes. Langhals and Jona, U.S. Pat. No. 6,166,210, describes perylene imide monocarboxylic acids for use as colorants. Langhals and Ismael, U.S. Pat. No. 6,143,890, teaches that perylenehydrazamides absorb at longer wavelength and exhibit increased lightfastness as compared to perylenebisimides. Langhals and Gold, U.S. Pat. No. 5,929,239 describes bifluorophoric perylene colorants. Hao et al., U.S. Pat. Nos. 5,874,580, 5,886,160, 6,013,776, 6,013,777, and 6,127,549 describe carbamate derivatives of various chromophores, including perylene.

Perylene dyes of various colors and light-absorbing properties have been reported. For example, Becker S. et al, Chem. Eur. J., 6, 21, 3984,(2000) report the synthesis of thermotropic perylenedicarboximide chromophores that show a color change from blue to orange. Holtrup F. O. et al, Tetrahedron, 53, 20, 6847, (1997) report the synthesis of purple benzoylperyleneimides that exhibit high thermal and photochemical stability. Langhals H. and Jona W., Angew. Chem. Int. Ed., 37,7,952 (1998)show the synthesis of bi- and trichromophoric perylene-3, 4:9,10-bis(dicarboximide)s with increased fluorescent properties. Zhao Y. and Wasielewski M. R., Tetrahedron Letters, 40, 7047, (1999), report the synthesis of a dialkylamino modified perylene dye that affords green products that exhibit intense optical absorption bands at 700 nm wavelength.

Unlike a variety of common fluorophores, however, the perylene chromophore has rarely been used as a biomolecular probe, apparently due to the strongly hydrophobic character of the molecule and difficulties with regiospecific labeling of biomolecules. Balakin K. V. et al., Nucleosides & Nucleotides, 18, 1279 (1999), report the synthesis of oligodeoxynucleotides bearing a 3′-terminal perylene-containing pseudonucleoside. The authors describe the use of these oligodeoxynucleotides as hybridization probes, which are detected by fluorescence anisotropy. Balakin K. V. et al. Biosensors & Bioelectronics 13, 771, (1998), describes the synthesis of a 3′-mer containing a 5′-(3-perylene)acetic acid residue, which shows no response in the fluorescence spectrum upon hybridization to the complementary sequence. Bevers S. A. et al, JACS, 122(25); 11004, (1998), and JACS, 120(42); 5905, (2000) report the synthesis of naphthalene- and perylene-based linkers for the stabilization of hairpin triplexes, and for duplex and triplex stabilization. However, the use of perylene dyes for the visual detection of biomolecules has not been described.

There is thus a need in the art for biomarkers that permit visual detection of biomolecules without prior illumination or chemical or enzymatic activation. Ideally, such biomarkers should be intensely colored and should be available in a variety of colors.

### BRIEF SUMMARY OF THE INVENTION

The present invention provides visually detectable biomolecules and reagents for their preparation, as well as methods for visually detecting a biomolecule and for determining the size of a biomolecule. The labeled biomolecules of the invention are intensely colored and can be readily observed by visual inspection, without prior illumination or chemical or enzymatic activation. By appropriate selection of the biomarker, as described herein, visually detectable biomolecules of a variety of colors may be obtained.

In a first aspect, therefore, the invention provides a visually detectable biomolecule of formula B—(—L—(D)ₘ)ₙ,

m and n are each an integer from one to about 5;

B is a biomolecule;

L, at each occurrence, is a spacer group comprising from one to about 10 linear atoms,

where L is attached to B by means of an ester, amide, phosphate, phosphorothioate, phosphonate, thioester, or disulfide linkage, and where the remaining linear atoms in L are selected from the group consisting of carbon, oxygen, nitrogen, and sulfur;

wherein the linear atoms in L can be optionally substituted and optionally can be included in a ring; and

D, at each occurrence, is a radical of a photostable visible dye, wherein each D has one and only one linkage to a biomolecule, provided that D is not unsubstituted perylenyl.

In a preferred embodiment, the visually detectable biomolecule has the formula B—(—L—(P)ₘ)ₙ, wherein

m and n are each an integer from one to about 5;

B is a biomolecule;

L, at each occurrence, is a spacer group comprising from one to about 10 linear atoms,

where L is attached to B by means of an ester, amide, phosphate, phosphorothioate, phosphonate, thioester, or disulfide linkage, and where the remaining linear atoms in L are selected from the group consisting of carbon, oxygen, nitrogen, and sulfur;

wherein the linear atoms in L can be optionally substituted and optionally can be included in a ring; and

P, at each occurrence, is a radical of a perylene, anthracene, naphthalene, or pyrene derivative, wherein each P has one and only one linkage to a biomolecule.

In another preferred embodiment according to this aspect of the invention, the visually detectable biomolecule has a defined molecular weight, and is useful as a molecular weight standard. The invention further provides a kit for determining the size of a test biomolecule, comprising a collection of two or more such visually detectable biomolecules of defined molecular weight.

In another aspect, therefore, the invention provides a method for determining the size of a test biomolecule, comprising (a) subjecting a visually detectable biomolecule kit according to the invention to conditions under which the biomolecule standards migrate to different positions according to molecular weight, thereby producing a visual ladder of biomolecule standards; (b) subjecting the test biomolecule to the same conditions employed in step (a); and (c) comparing the migration of the test biomolecule to the visual ladder of biomolecule standards to determine the molecular weight of the test biomolecule.

In another aspect, the invention provides a method for visually detecting a biomolecule, comprising: (a) providing a biological system with a visually detectable biomolecule of formula B—(—L—(D)ₘ)ₙ or B—(—L—(P)ₘ)ₙ, wherein B, L, D, P, m, and n are as defined above for the first aspect of the invention; and (b) detecting the biomolecule by its visible properties.

In yet another aspect, the invention provides reactive dyes for use in preparing the visually detectable biomolecules of the invention. In one embodiment, the reactive dye has the formula (D)ₙ—L—X, wherein

D is a radical of a photostable visible dye;

L is a spacer group comprising from one to about 10 linear atoms selected from the group consisting of carbon, oxygen, nitrogen, and sulfur;

wherein the linear atoms in L can be optionally substituted and optionally can be included in a ring; and

X is

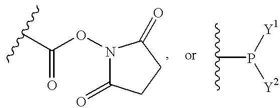

wherein Y¹ and Y² are independently dialkylamino, N-heterocyclic radical, or OZ, where Z is a protecting group.

In a preferred embodiment, the reactive dye has the formula (P)ₙ—L—X, wherein P is a radical of a derivative of perylene, anthracene, naphthalene, or pyrene, and n, L, and X are as described above for the reactive dye of formula (D)ₙ—L—X.

In another embodiment, the reactive dye has the formula(P)ₙ—L—X, wherein

P is a radical of a perylene derivative having a formula selected from the group consisting of:

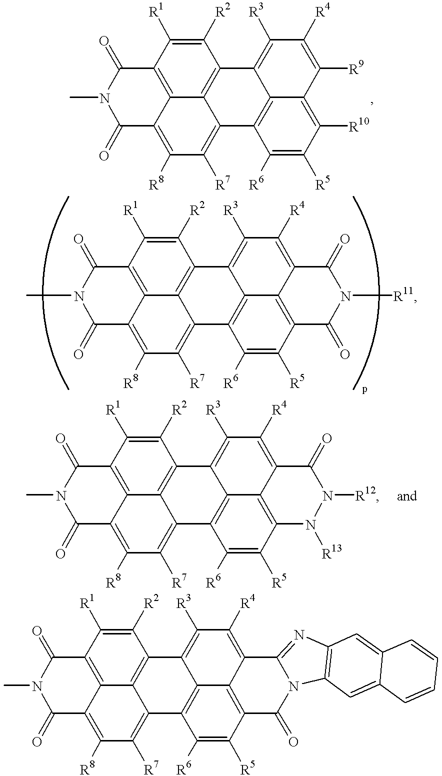

wherein

R¹, R², R³, R⁴, R⁵, R⁶, R⁷, R⁸, R⁹, and R¹⁰ are radicals independently selected from the group consisting of hydrogen, halogen, cyano, hydroxy, alkoxy, amino, alkylamino, dialkylamino, heterocyclic radical, alkyl, and aryl, wherein the alkyl and aryl groups may be optionally substituted, and wherein adjacent radicals can form a carbocyclic or heterocyclic ring;

p is 0, 1, or 2;

R¹¹ is C₁-C₆ alkyl, C₆-C₁₀ aryl, or (C₆-C₁₀)ar(C₁-C₆)alkyl;

R¹² and R¹³ are independently C₁-C₆ alkyl, C₆-C₁₀ aryl, or (C₆-C₁₀)ar(C₁-C₆)alkyl;

L is a spacer group comprising from one to about 10 linear atoms selected from the group consisting of carbon, oxygen, nitrogen, and sulfur;

wherein the linear atoms in L can be optionally substituted and optionally can be included in a ring; and

X is a reactive group that enables attachment to an amino, hydroxy, carboxyl, or sulfhydryl group on a biomolecule.

In another aspect, the invention provides a method for visually detecting a biomolecule, comprising contacting a biomolecule with a reactive dye of formula (D)ₙ—L—X, wherein

D is a radical of a photostable visible dye;

L is a spacer group comprising from one to about 10 linear atoms selected from the group consisting of carbon, oxygen, nitrogen, and sulfur;

wherein the linear atoms in L can be optionally substituted and optionally can be included in a ring; and

X is a reactive group that enables attachment to an amino, hydroxy, carboxyl, or sulfhydryl group on a biomolecule;

whereby a visually detectable biomolecule of formula B—(—L—(D)ₘ)ₙ is produced; and

detecting the biomolecule by its visible properties.

In a preferred embodiment according to this aspect of the invention, the reactive dye has the formula (P)ₙ—L—X, wherein

P is a radical of a perylene, anthracene, naphthalene, or pyrene derivative;

L is a spacer group comprising from one to about 10 linear atoms selected from the group consisting of carbon, oxygen, nitrogen, and sulfur;

wherein the linear atoms in L can be optionally substituted and optionally can be included in a ring; and

X is a reactive group that enables attachment to an amino, hydroxy, carboxyl, or sulfhydryl group on a biomolecule.

### DETAILED DESCRIPTION OF THE PREFERRED EMBODIMENTS

The present invention provides methods for detecting biomolecules that are an alternative to currently employed methods that rely on fluorescent, chemiluminescent, or radioactive biomarkers. In particular, the invention provides visually detectable biomolecules and reagents for their preparation, as well as methods for visually detecting a biomolecule and for determining the size of a biomolecule.

The patent and scientific literature referred to herein establishes knowledge that is available to those with skill in the art. The issued patents, applications, and references that are cited herein are hereby incorporated by reference to the same extent as if each was specifically and individually indicated to be incorporated by reference. In the case of inconsistencies, the present disclosure will prevail.

In a first aspect, the invention provides a visually detectable biomolecule of formula B—(—L—(D)ₘ)ₙ,

m and n are each an integer from one to about 5;

B is a biomolecule;

L, at each occurrence, is a spacer group comprising from one to about linear atoms,

where L is attached to B by means of an ester, amide, phosphate, phosphorothioate, phosphonate, thioester, or disulfide linkage, and where the remaining linear atoms in L are selected from the group consisting of carbon, oxygen, nitrogen, and sulfur;

wherein the linear atoms in L can be optionally substituted and optionally can be included in a ring; and

D, at each occurrence, is a radical of a photostable visible dye, wherein each D has one and only one linkage to a biomolecule.

For purposes of the present invention, the term “biomolecule” refers to any of a variety of biological materials, including nucleic acids, carbohydrates, amino acids, polypeptides, glycoproteins, hormones, and mixtures thereof. More specifically, the term is intended to include, without limitation, RNA, DNA, oligonucleotides, modified or derivatized nucleotides, enzymes, receptors, receptor ligands (including hormones), antibodies, antigens, and toxins, as well as bacteria, viruses, blood cells, and tissue cells. The visually detectable biomolecules of the invention are prepared, as further described herein, by contacting a biomolecule with a visible dye having a reactive group that enables attachment to an amino, hydroxy, carboxyl, or sulfhydryl group on the biomolecule.

The terms “visible” and “visually detectable” are used herein to refer to substances that are observable by visual inspection, without prior illumination, or chemical or enzymatic activation. Such visually detectable substances absorb and emit light in a region of the spectrum ranging from about 400 to about 800 nm. Preferably, such substances are intensely colored, preferably having a molar extinction coefficient of at least about 40,000, more preferably at least about 50,000, still more preferably at least about 60,000, yet still more preferably at least about 70,000, and most preferably at least about 80,000 M⁻¹ cm⁻¹. The biomolecules of the invention may be detected by observation with the naked eye, or with the aid of a optically based detection device, including, without limitation, absorption spectrophotometers, transmission light microscopes, digital cameras and scanners.

L is a spacer group between the biomolecule, B, and the visible dye, D. The structure of L is not critical, so long as it does not interfere with the function of B or prevent detection of the visible chromophore D. Preferably, L comprises from one to about 10 linear atoms, where L is attached to a ring atom in D and is attached to B by means of an ester amide, phosphate, phosphorothioate, phosphonate, thioester, or disulfide linkage. The remaining linear atoms in L are preferably selected from the group consisting of carbon, oxygen, nitrogen and sulfur, any of which atoms optionally may be included in a carbocyclic or heterocyclic ring. The linear carbon atoms in L optionally can be substituted with a substituent selected from the group consisting of halo, hydroxy, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido. A linear nitrogen atom in L optionally can be substituted with acyl, sulfonyl, alkyl, alkaryl, aryl, aralkyl, alkoxycarbonyl. A linear sulfur atom in L optionally can be oxidized.

In some embodiments, the biomolecule is preferably attached to more than one dye molecule in order to enhance the sensitivity of detection. However, each P has only one linkage to a biomolecule. To further enhance sensitivity, L may comprise a dendrimer. For purposes of the invention, the term “dendrimer” refers to a structure having multiple arms so that more than one dye molecule may be attached to a single spacer group. An example of a biomolecule of the invention comprising such a dendrimer structure is shown below:

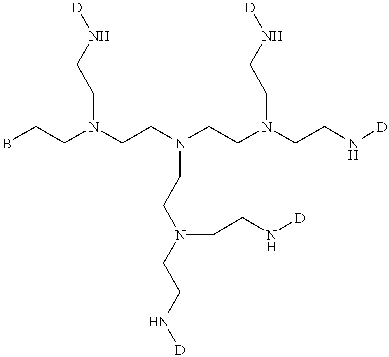

For purposes of the invention, the term “photostable visible dye” refers to a chemical moiety that is visually detectable, as defined hereinabove, and is not significantly altered or decomposed upon exposure to light. Preferably, the photostable visible dye does not exhibit significant bleaching or decomposition after being exposed to light for at least one hour. More preferably, the visible dye is stable after exposure to light for at least 12 hours, still more preferably at least 24 hours, still yet more preferably at least one week, and most preferably at least one month. Nonlimiting examples of photostable visible dyes suitable for use in the compounds and methods of the invention include azo dyes, thioindigo dyes, quinacridone pigments, dioxazine, phthalocyanine, perinone, diketopyrrolopyrrole, quinophthalone, and truarycarbonium.

In a preferred embodiment, the visually detectable biomolecule has the formula B—(—L—(P)ₘ)ₙ, wherein

m and n are each an integer from one to about 5;

B is a biomolecule;

L, at each occurrence, is a spacer group comprising from one to about 10 linear atoms,

where L is attached to B by means of an ester, amide, phosphate, phosphorothioate, phosphonate, thioester, or disulfide linkage, and where the remaining linear atoms in L are selected from the group consisting of carbon, oxygen, nitrogen, and sulfur;

wherein the linear atoms in L can be optionally substituted and optionally can be included in a ring; and

P, at each occurrence, is a radical of a perylene, anthracene, naphthalene, or pyrene derivative, wherein each P has one and only one linkage to a biomolecule.

As used herein, the term “perylene derivative” is intended to include any substituted perylene that is visually detectable. However, the term is not intended to include perylene itself. The terms “anthracene derivative”, “naphthalene derivative”, and “pyrene derivative” are used analogously.

In some preferred embodiments, P is an imide, bisimide or hydrazamimide derivative of perylene, anthracene, naphthalene, or pyrene. Preferably, P is a perylene imide, perylene bisimide, or perylene hydrazamimide, wherein L is preferably attached to the imide nitrogen. In certain preferred embodiments, P has a formula selected from the group consisting of:

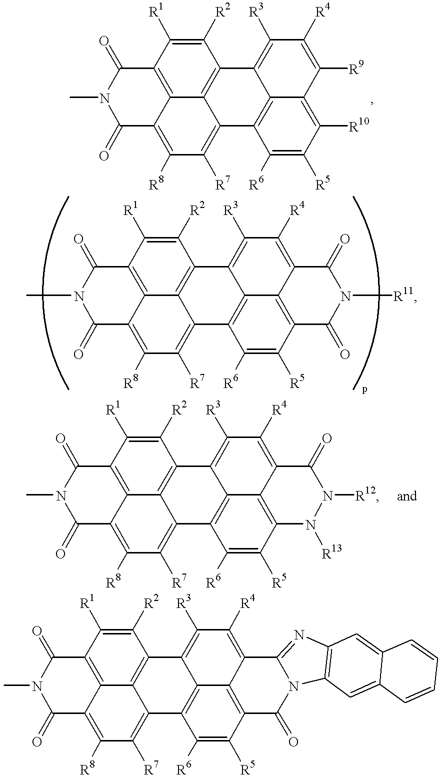

wherein

R¹, R², R³, R⁴, R⁵, R⁶, R⁷, R⁸, R⁹, and R¹⁰ are radicals independently selected from the group consisting of hydrogen, halogen, cyano, hydroxy, alkoxy, amino, alkylamino, dialkylamino, heterocyclic radical, alkyl, and aryl, wherein the alkyl and aryl groups may be optionally substituted, and wherein adjacent radicals can form a carbocyclic or heterocyclic ring;

p is 0, 1, or 2;

R¹¹ is C₁-C₆ alkyl, C₆-C₁₀ aryl, or (C₆-C₁₀)ar(C₁-C₆)alkyl;

R¹² and R¹³ are independently C₁-C₆ alkyl, C₆-C₁₀ aryl, or (C₆-C₁₀)ar(C₁-C₆)alkyl;

In one preferred embodiment according to this aspect of the invention, the visually detectable biomolecule has a defined molecular weight, and is useful as a molecular weight standard. The invention further provides a kit for determining the size of a test biomolecule, comprising a collection of two or more such visually detectable biomolecules of defined molecular weight. When subjected to electrophoresis, the biomolecule standards migrate to different positions according to molecular weight, thereby producing a visual biomolecule ladder.

Biomolecule ladders, such as DNA ladders, are common reagents for determining the size of test biomolecules. Thus, in another aspect, the invention provides a method for determining the size of a test biomolecule, comprising (a) subjecting a visually detectable biomolecule kit according to the invention to conditions under which the biomolecule standards migrate to different positions according to molecular weight, thereby producing a visual ladder of biomolecule standards; (b) subjecting the test biomolecule to the same conditions employed in step (a); and (c) comparing the migration of the test biomolecule to the visual ladder of biomolecule standards to determine the molecular weight of the test biomolecule.

Any conditions suitable for separating molecules according to molecular weight may be used for practice of the method according to this aspect of the invention. Nonlimiting examples of such separation conditions include chromatographic and electrophoretic techniques. Typically, a biomolecule test sample, such as a DNA sample, and a biomolecule ladder are loaded in adjacent wells on an electrophoresis gel, such as an agarose or polyacrylamide gel. The sample is separated by electrophoresis through the gel, and the size of the test biomolecule is determined by comparing its migration with the bands of known size in the biomolecule ladder. Currently available ladders require additional steps for visualization. By contrast, the biomolecule ladders of the present invention are immediately visible to the naked eye, even while they are still running in the gel.

More generally, visible biomarkers can be utilized as purification tools, obviating the need for analytical or biochemical assaying of fractions. Currently available biomarkers require additional steps for visualization or are unstable to light for the time periods necessary to effect separation. By contrast, the visually detectable biomolecules of the invention can be simply observed by visual inspection during analytical or preparative separations. In certain preferred embodiments, chromatographic methods are used to separate the biomolecules. Such chromatographic methods may include, without limitation, paper chromatography, thin layer chromatography, including preparative thin layer chromatography, and column chromatography. Nonlimiting examples of suitable chromatography conditions include normal phase and reverse phase silica gel chromatography, size exclusion chromatography, and ion exchange chromatography. In certain other preferred embodiments, art-recognized electrophoretic methods are used to separate the biomolecules. It will be apparent to one skilled in the art that the chromatographic or electrophoretic conditions may be selected and modified to achieve optimum separation and/or purification of the biomolecule of interest.

The visually detectable biomolecules of the invention are also useful for a wide variety of biochemical and biomedical applications in which there is a need to determine the presence, location, or quantity of a particular biomolecule. In another aspect, therefore, the invention provides a method for visually detecting a biomolecule, comprising: (a) providing a biological system with a visually detectable biomolecule of formula B—(—L—(D)ₘ)ₙ, or B—(—L—(P)ₘ)ₙ, wherein B, L, D, P, m, and n are as described above for the first aspect of the invention; and (b) detecting the biomolecule by its visible properties.

For purposes of the invention, the phrase “detecting the biomolecule by its visible properties” means that the biomolecule, without illumination or chemical or enzymatic activation, is observed with the naked eye, or with the aid of a optically based detection device, including, without limitation, absorption spectrophotometers, transmission light microscopes, digital cameras and scanners. A densitometer may be used to quantify the amount of visually detectable biomolecule present. For example, the relative quantity of the biomolecule in two samples can be determined by measuring relative optical density. If the stoichiometry of dye molecules per biomolecule is known, and the extinction coefficient of the dye molecule is known, then the absolute concentration of the biomolecule can also be determined from a measurement of optical density.

As used herein, the term “biological system” is used to refer to any solution or mixture comprising one or more biomolecules in addition to the visually detectable biomolecule. Nonlimiting examples of such biological systems include cells, cell extracts, tissue samples, electrophoretic gels, assay mixtures, and hybridization reaction mixtures.

The methods of the invention are useful for microarray and high-throughput screening applications. Microarray technologies have successfully allowed for the rapid study of vast numbers of genes, and have become an indispensable tool for molecular biologists studying the genome. MacBeath G. et al., Science 289: 1760 (2000) describes protein microarrays, wherein proteins are arrayed on a glass surface. Nyquist R. M. et al., Langmuir, 16: 1793 (2000) describes efforts to apply this technology to carbohydrates. The visually detectable biomolecules of the invention permit rapid and inexpensive detection of binding in microarrays, including protein, nucleic acid, carbohydrate, and glycoprotein microarrays.

In some embodiments, the visually detectable biomolecule B—(—L—(D)ₘ)ₙ or B—(—L—(P)ₘ)ₙ of the invention is used in a two-step detection process. In these embodiments, the visually detectable biomolecule is used as a probe to detect a second component in the biological system, to which it specifically binds. Preferably, the biological system is contacted with the visually detectable biomolecule to permit binding of the biomolecule to the second component. The mixture is preferably washed to remove visually detectable biomolecules that are nonspecifically bound. The intensity of color is then indicative of the amount of the second component present in the biological system.

For example, in one particularly preferred embodiment, B is streptavidin, and the visually detectable biomolecule is used as a probe to detect a biotin-labeled component in the biological system. Likewise, a streptavidin-labeled component in the biological system can be detected with a visually detectable biomolecule in which B is biotin. It will thus be apparent to those of skill in the art that the visually detectable biomolecules of the invention are readily adapted for use in sandwich assays.

In another particularly preferred embodiment, B is an antibody, preferably a monoclonal antibody, and the visually detectable biomolecule can be used to detect an antigen for which the antibody is specific. For example, the visually detectable antibody can be used in biological assays, such as sandwich assays. The visually detectable antibody can also be used to detect cell surface antigens, and can be used for cell sorting or for cell staining in tissue samples. The method can also be used to determine the density of cell surface antigens, such as cell surface receptors.

In certain preferred embodiments, the method employs a plurality of visually detectable biomolecules of the invention, each B being specific for a different component in the biological system. Preferably, each D exhibits different visible properties, e.g., absorption spectrum. More preferably, each of the plurality of visually detectable biomolecules has a different color. Thus, the method according to this embodiment permits multiple components in a biological system to be simultaneously visualized and distinguished.

In another particularly preferred embodiment, B is a nucleic acid, and the visually detectable biomolecule is used as a probe to detect a nucleic acid component in the biological system having a complementary sequence. The biological system is preferably contacted with the visually detectable nucleic acid probe according to any of the standard hybridization conditions known in the art. Both the probe and the target nucleic acid may comprise RNA, DNA, modified nucleic acids, or combinations thereof.

The visually detectable biomolecules of the invention may be prepared by any suitable method. Typically, the biomolecule is contacted with a reactive dye reagent having a reactive group that enables attachment to an amino, hydroxy, carboxyl, or sulfhydryl group on a biomolecule.

In another aspect, therefore, the invention provides reactive dyes for use in preparing the visually detectable biomolecules of the invention. In one embodiment, the reactive dye has the formula (D)ₙ—L—X, wherein

D is a radical of a photostable visible dye;

L is a spacer group comprising from one to about 10 linear atoms selected from the group consisting of carbon, oxygen, nitrogen, and sulfur;

wherein the linear atoms in L can be optionally substituted and optionally can be included in a ring; and

X is

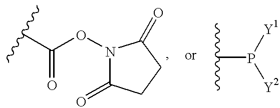

wherein Y¹ and Y² are independently dialkylamino, N-heterocyclic radical, or OZ, where Z is a protecting group.

In some preferred embodiments, X is an electrophilic moiety that enables attachment to a nucleophilic amino, hydroxy, or sulfhydryl group on the biomolecule.

When the biomolecule to which the reactive dye is to be attached is a nucleic acid, the reactive group X preferably has the formula —P(Y¹)(Y²), which enables attachment to a hydroxy group on the nucleic acid. Preferably, Y¹ is OZ, where Z is preferably selected from the group consisting of alkyl, allyl, aryl, or cyanoalkyl, and Y² is dialkylamino. Most preferably, Y¹ is cyanoethyl and Y² is diisopropylamino. In these embodiments, standard phosphoramidite chemistry is employed to attach the dye to the biomolecule.

When the reactive dye is to be attached to an amino or hydroxy group of a biomolecule other than a nucleic acid, X is preferably an isocyanate, isothiocyanate, dichlorotriazine, or activated ester, such as an N-hydroxysuccinimide ester. When the reactive dye is to be attached to a sulfhydryl group on the biomolecule, X is preferably an activated disulfide moiety, such as a pyridyldisulfide.

In some other preferred embodiments, X is a nucleophilic moiety that enables attachment to a carboxyl group on the biomolecule. In these embodiments, X is preferably hydroxy or amino. Reaction of X with a carboxyl group on the biomolecule can be effected by treatment with a coupling reagent, such as ethylcarbodiimide hydrochloride (EDC.HCl). Alternatively, the carboxyl group can be activated prior to reaction with X, for example by conversion to an activated ester moiety such as an N-hydroxysuccinimide ester.

In a preferred embodiment, the reactive dye has the formula (P)ₙ—L—X, wherein

P is a radical of a perylene, anthracene, naphthalene, or pyrene derivative;

L is a spacer group comprising from one to about 10 linear atoms selected from the group consisting of carbon, oxygen, nitrogen, and sulfur;

wherein the linear atoms in L can be optionally substituted and optionally can be included in a ring; and

X is

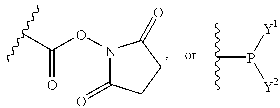

wherein Y¹ and Y² are independently dialkylamino, N-heterocyclic radical, or OZ, where Z is a protecting group.

In another embodiment, the reactive dye has the formula (P)ₙ—L—X, wherein

P is a radical of a perylene derivative having a formula selected from the group consisting of:

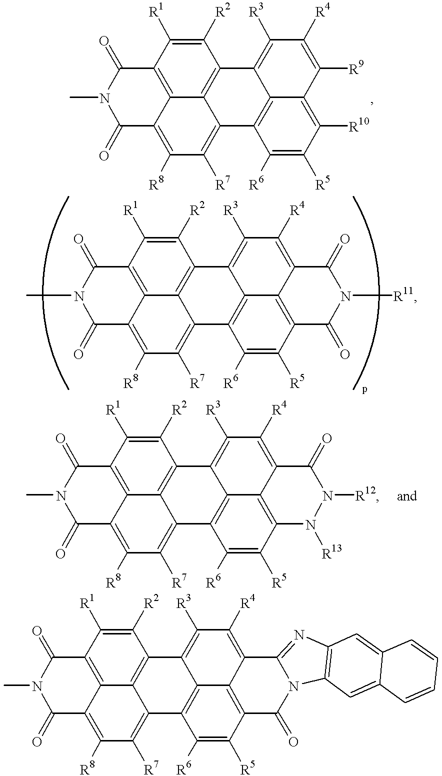

wherein

R¹, R², R³, R⁴, R⁵, R⁶, R⁷, R⁸, R⁹ and R¹⁰ are radicals independently selected from the group consisting of hydrogen, halogen, cyano, hydroxy, alkoxy, amino, alkylamino, dialkylamino, heterocyclic radical, alkyl, and aryl, wherein the alkyl and aryl groups may be optionally substituted, and

wherein adjacent radicals can form a carbocyclic or heterocyclic ring;

p is 0, 1, or 2;

R¹¹ is C₁-C₆ alkyl, C₆-C₁₀ aryl, or (C₆-C₁₀)ar(C₁-C₆)alkyl;

R¹² and R¹³ are independently C₁-C₆ alkyl, C₆-C₁₀ aryl, or (C₆-C₁₀)ar(C₁-C₆)alkyl;

L is a spacer group comprising from one to about 10 linear atoms selected from the group consisting of carbon, oxygen, nitrogen, and sulfur;

wherein the linear atoms in L can be optionally substituted and optionally can be included in a ring; and

X is a reactive group that enables attachment to an amino, hydroxy, carboxyl, or sulfhydryl group on a biomolecule.

In yet another aspect, the invention provides a method for visually detecting a biomolecule, comprising contacting a biomolecule with a reactive dye of formula (D)ₙ—L—X or (P)ₙ—L—X, as defined above, whereby a visually detectable biomolecule of formula B—(—L—(D)ₘ)ₙ or B—(—L—(P)ₘ)ₙ is produced; and detecting the biomolecule by its visible properties.

Definitions

The term “alkyl” as employed herein refers to straight and branched chain aliphatic groups having from 1 to 12 carbon atoms, preferably 1-8 carbon atoms, and more preferably 1-6 carbon atoms, which may be optionally substituted with one, two or three substituents. Unless otherwise apparent from context, the term “alkyl” is meant to include saturated, unsaturated, and partially unsaturated aliphatic groups. Preferred saturated alkyl groups include, without limitation, methyl, ethyl, propyl, isopropyl, butyl, isobutyl, sec-butyl, tert-butyl, pentyl, and hexyl. The term “carbocyclic group” as employed herein includes saturated and partially unsaturated cyclic hydrocarbon groups having 3 to 12 carbons, preferably 3 to 8 carbons, and more preferably 3 to 6 carbons, wherein the cycloalkyl group

additionally may be optionally substituted. Preferred carbocyclic groups include, without limitation, cyclopropyl, cyclobutyl, cyclopentyl, cyclopentenyl, cyclohexyl, cyclohexenyl, cycloheptyl, and cyclooctyl.

An “aryl” group is a C₆-C₁₄ aromatic moiety comprising one to three aromatic rings, which may be optionally substituted. Preferably, the aryl group is a C₆-C₁₀ aryl group. Preferred aryl groups include, without limitation, phenyl, naphthyl, anthracenyl, and fluorenyl. An “aralkyl” or “arylalkyl” group comprises an aryl group covalently linked to an alkyl group, either of which may independently be optionally substituted or unsubstituted. Preferably, the aralkyl group is (C₁-C₆)alk(C₆-C₁₀)aryl, including, without limitation, benzyl, phenethyl, and naphthylmethyl. An “alkaryl” or “alkylaryl” group is an aryl group having one or more alkyl substituents. Examples of alkaryl groups include, without limitation, tolyl, xylyl, mesityl, ethylphenyl, tert-butylphenyl, and methylnaphthyl.

A “heterocyclic” group is a ring structure having from about 3 to about 8 atoms, wherein one or more atoms are selected from the group consisting of N, O, and S. The heterocyclic group may be optionally substituted on carbon at one or more positions. The heterocyclic group may also independently be substituted on nitrogen with alkyl, aryl, aralkyl, alkylcarbonyl, alkylsulfonyl, arylcarbonyl, arylsulfonyl, alkoxycarbonyl, aralkoxycarbonyl, or on sulfur with oxo or lower alkyl. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino. In certain preferred embodiments, the heterocyclic group is fused to an aryl or heteroaryl group. Examples of such fused heterocyles include, without limitation, tetrahydroquinoline and dihydrobenzofuran.

As used herein, the term “heteroaryl” refers to groups having 5 to 14 ring atoms, preferably 5, 6, 9, or 10 ring atoms; having 6, 10, or 14 π electrons shared in a cyclic array; and having, in addition to carbon atoms, between one and about three heteroatoms selected from the group consisting of N, O, and S. Preferred heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

As employed herein, a “substituted” alkyl, cycloalkyl, aryl, heteroaryl, or heterocyclic group is one having between one and about four, preferably between one and about three, more preferably one or two, non-hydrogen substituents. Suitable substituents include, without limitation, halo, hydroxy, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

The term “halogen” or “halo” as employed herein refers to chlorine, bromine, fluorine, or iodine.

As herein employed, the term “acyl” refers to an alkylcarbonyl or arylcarbonyl substituent.

The term “acylamino” refers to an amide group attached at the nitrogen atom. The term “carbamoyl” refers to an amide group attached at the carbonyl carbon atom. The nitrogen atom of an acylamino or carbamoyl substituent may be additionally substituted. The term “sulfonamido” refers to a sulfonamide substituent attached by either the sulfur or the nitrogen atom. The term “amino” is meant to include NH₂, alkylamino, arylamino, and cyclic amino groups.

The term “ureido” as employed herein refers to a substituted or unsubstituted urea moiety.

### EXAMPLES

### Example 1

### Synthesis of Reactive Dye I N-2{[2-O-(2-cyanoethyl diisopropylchlorophosphino)ethoxy]ethyl}N′-2-methyl-3,4,9,10,-Perylenetetracarboxylic Diimide

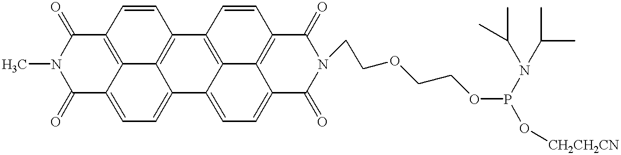

### 2-(2-O-tButyldimethylsilylethoxy)ethylamine (A).

To 5.0 g (48 mmol) of 2-(2-aminoethoxy)ethanol dissolved in anhydrous pyridine (50 mL) was added 7.2 g (48 mmol) tert-butyldimethylsilyl chloride and 4.9 g (72 mmol) of imidazole. The reaction mixture was stirred overnight, until TLC analysis indicated the absence of starting material and the formation of new products. Pyridine was removed in vacuo. The mixture was dissolved in dichloromethane (50 mL) and was washed with an aqueous solution of saturated sodium bicarbonate (2×50 mL). The organic layer was dried over anhydrous sodium sulfate. The mixture was then vacuum filtered, the filter was washed with dichloromethane, and the product was purified by flash chromatography to yield (A).

N-2-[(2-OtButyldimethylsilylethoxy)ethyl]perylene-3,4:9,10-tetracarboxylic-3,4-anhydride-9,10-carboximide (B)

To 2.0 g (5.1 mmol) perylenetetracarboxylic dianhydride suspended in anhydrous pyridine or quinoline (25 mL) was added 1.16 g (5.1 mmol) 2-(2-O-t-butyldimethylsilylethoxy)ethylamine (A) and 1.1 g (5.1 mmol) zinc acetate dihydrate. The reaction mixture was refluxed overnight, until TLC analysis indicated the absence of starting material and the formation of products. Pyridine was removed in vacuo. The mixture was suspended in chloroform and loaded onto a large silica column. The product was purified by flash chromatography to give (B) as a dark red solid (50-90% yield). Other possible solvents include toluene, m-cresol, and N-methylpyrolidone. A capping reagent such as methylamine or benzylamine (but not limited to these two) can be attached to the other end of the perylene moiety in the following manner:

N-2-[(2-OtButyldimethylsilylethoxy)ethyl]N′-2-methyl-3,4:9,10-perylene-tetracarboxylic diimide (C).

To 2.0 g (5.1 mmol) N-2-[(2-OtButyldimethylsilylethoxy)ethyl]perylene-3,4:9,10-tetracarboxylic-3,4-anhydride-9,10-carboximide (B) suspended in anhydrous pyridine or quinoline (25 mL) is added 5.1 mmol of methylamine and 5.1 mmol of zinc acetate dihydrate. The reaction mixture is refluxed overnight, until TLC analysis indicates the absence of starting material and the formation of products. Pyridine is removed in vacuo. The mixture is suspended in chloroform, loaded onto a large silica column, and purified by flash chromatography to give (C) as a dark red solid (50-90% yield).

N-2-[(2-hydroxyethoxy)ethyl]-N′-2-methyl-3,4,9,10,-perylenetetracarboxylic diimide (D).

To 4.2 mmol of N-2-[(2-OtButyldimethylsilylethoxy)ethyl]N′-2-methyl-3,4,9,10-perylenetetracarboxylic diimide (C) dissolved in anhydrous pyridine (50 mL) is added HF/Pyridine (70%) until the starting material is consumed, as monitored by TLC analysis. Pyridine is removed in vacuo. The mixture is resuspended in chloroform and purified by flash chromatography to yield 50-90% of compound (D).

N-2-{[2-O-(2-cyanoethyl diisopropylchlorophosphino)ethoxy]ethyl}N′-2-methyl-3,4,9,10,-perylenetetracarboxylic diimide (Reactive Dye I).

To 0.055 mmol of N-2-[(2-hydroxyethoxy)ethyl]-N′-2-methyl-3,4,9,10,-perylenetetracarboxylic diimide (D) dissolved in anhydrous dichloromethane (1 mL) at 0° C. is added 0.048 mL (0.28 mmol) diisopropylethylamine followed by 0.025 mL (0.11 mmol) 2-cyanoethyl diisopropylchlorophosphoramidite. The reaction is slowly brought to 25° C. and stirred for 30 minutes. When TLC analysis indicates the absence of starting material and the presence of a new product, the reaction is stopped with 2-3 drops of methanol, and the phosphitylated product is precipitated into cold hexane (0° C.). The product is filtered, washed with cold hexane and rinsed from the filter with dichloromethane. The product is concentrated to yield 50-90 molar percent of Reactive Dye I as a red solid.

### Example 2

### Synthesis of Reactive Dye II: 1,7-dipyrolo-N-2-{[2-O-(2-cyanoethyl diisopropylchlorophosphino)ethoxy]ethyl}N′-2-methyl-3,4,9,10,-perylenetetracarboxylic Diimide

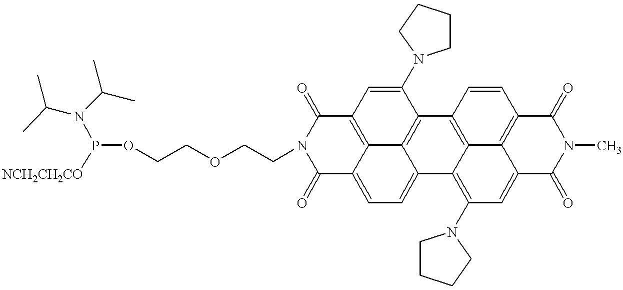

### 2-(2-O-tButyldimethylsilylethoxy)ethylamine (A).

To 5.0 g (48 mmol) of 2-(2-aminoethoxy)ethanol dissolved in anhydrous pyridine (50 mL) was added 7.2 g (48 mmol) tert-butyldimethylsilyl chloride and 4.9 g (72 mmol) of imidazole. The reaction mixture was stirred overnight, until TLC analysis indicated the absence of starting material and formation of new products. Pyridine was removed in vacuo. The mixture was dissolved in dichloromethane (50 mL), washed with an aqueous solution of saturated sodium bicarbonate (2×50 mL), and dried over anhydrous sodium sulfate. The mixture was then vacuum filtered and the filter was washed with dichloromethane. The product was purified by flash chromatography to yield 3.5 g (16 mmol) of (A) as brown oil (36% yield).

1,7-dibromo-3,4,9,10-Perylenetetracarboxylicdianhydride (E).

The starting material, 1,7-dibromoperylene-3,4,9,10-tetracarboxylic dianhydride, can be obtained by selective bromination of perylene-3,4,9,10-tetracarboxylic dianhydride in 100% by weight sulfuric acid (monohydrate), as described by Bohm A. et al., Patent No. DE19547210. An expedient procedure comprises first stirring perylene-3,4,9,10-tetracarboxylic dianhydride in the sulfuric acid for 2-6 hours and then heating this mixture, after adding a halogenation catalyst such as iodine (preferably 30-40 mmol per mole of anhydride) to the reaction temperature (generally 80-90° C.). At this point, the bromine is added slowly dropwise (usually over 6-10 hours), preferably using 2-2.5 mol of bromine (Br₂) per mole of anhydride. After cooling to room temperature and displacing the unreacted bromine by nitrogen, water is added, a little at a time, in order to reduce the concentration of sulfuric acid to about 85-88% by weight. Working up the reaction mixture to the 1,7-dibromoperylene-3,4,9,10-tetracarboxylic dianhydride (E) can be carried out by filtering off the precipitated product, washing it with 85-88% by weight sulfuric acid, stirring it into water, filtering the mixture again, washing the product with water and then drying it.

1,7-dibromo-N-2-[(2-OtButyldimethylsilylethoxy)ethyl]perylene-3,4:9,10-tetracarboxylic-3,4-anhydride-9,10-carboximide (F).

To 5.1 mmol of 1,7-dibromo-perelynetetracarboxylicdianhydride (E) suspended in anhydrous pyridine or quinoline (25 mL) is added 5.1 mmol of 2-(2-O-t-butyl-dimethylsilylethoxy)ethylamine (A) and 5.1 mmol zinc acetate dihydrate. The reaction mixture is refluxed overnight, until TLC analysis indicates the absence of starting material and the formation of products. Pyridine is removed in vacuo. The mixture is suspended in chloroform, loaded onto a large silica column, and purified by flash chromatography to give (E) as a dark red solid (50-90% yield). Other possible solvents include toluene, m-cresol, N-methylpyrolidone. A capping reagent such as methylamine or benzylamine (but not limited to these two) can be attached to the other end of the perylene moiety in the following manner:

1,7-dibromo-N-2-[(2-OtButyldimethylsilylethoxy)ethyl]N′-2-methyl-3,4,9,10-Perylenetetracarboxylic Diimide (G).

To 5.1 mmol 1,7-dibromo-N′-2-[(2-OtButyldimethylsilylethoxy)ethyl]imide-3,4,9,10-Perylenetetracarboxylic anhydride (E) suspended in anhydrous pyridine or quinoline (25 mL) is added 5.1 mmol methylamine and 5.1 mmol zinc acetate dihydrate. The reaction mixture is refluxed overnight, until TLC analysis indicates the absence of starting material and the formation of products. Pyridine is removed in vacuo. The mixture is suspended in chloroform, loaded onto a large silica column, and purified by flash chromatography to give (G) as a dark red solid (50-90% yield).

1,7-dipyrolo-N-2-[(2-OtButyldimethylsilylethoxy)ethyl]N′-2-methyl-3,4,9,10-Perylenetetracarboxylic Diimide (H)

1,7-dibromo-N-2-[(2-OtButyldimethylsilylethoxy)ethyl]N′-2-methyl-3,4,9,10-perylenetetracarboxylic diimide (G) is dissolved in pyrrolidine. The solution is heated to 60° C. under dry nitrogen for 24 hours with stirring. Excess pyrrolidine is removed in vacuo and the residue is purified by column chromatography on silica to yield a green solid.

1,7-dipyrolo-N-2-[(2-hydroxyethoxy)ethyl]-N′-2-methyl-3,4,9,10,-perylenetetracarboxylic Diimide (I).

To 4.2 mmol 1,7-dipyrolo-N-2-[(2-OtButyldimethylsilylethoxy)ethyl]N′-2-methyl-3,4,9,10-perylenetetracarboxylic diimide (H) dissolved in anhydrous pyridine (50 mL) is added tetrabutylammonium fluoride until the silyl protecting group is cleaved, as monitored by TLC analysis. Pyridine is removed in vacuo. The mixture is resuspended in chloroform and purified by flash chromatography to yield 50-90% of compound (I).

1,7-dipyrolo-N-2-{[2-O-(2-cyanoethyl diisopropylchlorophosphino)ethoxy]ethyl}N′-2-methyl-3,4,9,10,-Perylenetetracarboxylic Diimide (Reactive Dye II).

To 0.055 mmol 1,7-dipyrolo-N-2-[(2-hydroxyethoxy)ethyl]-N′-2-methyl-3,4,9,10,-perylenetetracarboxylic diimide (F) dissolved in anhydrous dichloromethane (1 mL at 0° C.) is added 0.048 mL (0.28 mmol) diisopropylethylamine followed by 0.025 mL (0.11 mmol) 2-cyanoethyl diisopropylchlorophosphoramidite. The reaction is slowly brought to 25° C. and stirred for 30 minutes, until TLC analysis on alumina indicates the absence of starting material and the presence of a new product. The reaction is stopped with 2-3 drops of methanol and the phosphitylated product is precipitated into cold hexane (0° C.). The product is filtered, washed with cold hexane and rinsed from the filter with dichloromethane. The product is concentrated to yield 50-90 molar percent of Reactive Dye II.

### Example 3

### Synthesis of Reactive Dye III: N-2{[2-O-(2-cyanoethyl diisopropylchlorophosphino)-ethoxy]ethyl}-naptho[2′,3′:4,5]imidazo[1,2-b]anthra[2,1,9-def:6,5,10-d′,e′,f′]-diisoquinoline-1,3,16(2H)-trione

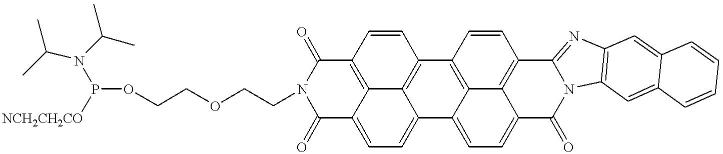

The dye is prepared according to Langhals H. et al., Liebigs Ann., 481, (1995), and the phosphoramidite is prepared according to the previously listed methods.

### Example 4

### Synthesis of Reactive Dye IV: N-2-[N-hydroxysuccinimidylpropanoyl]-naptho[2′,3′:4,5]imidazo[1,2-b]-anthra[2,1,9-d,e,f:6,5,10-d′,e′,f′]diisoquinoline-1,3,16(2H)-trione

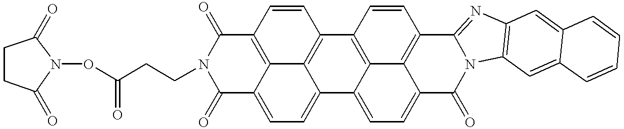

To a solution of N-hydroxysuccinimide (1 mmol, 1 equiv) and the carboxylic acid precursor (1 mmol, 1 equiv) in 7 mL diethyl ether is added dicyclohexylcarbodiimide (1 mmol, 1 equiv). The reaction mixture is stirred for 2 hr at room temperature and then filtered. Purification by column chromatography on silica gel affords Reactive Dye IV in 50-90% yield. The carboxylic acid precursor is prepared in accordance with procedures described by Langhals H., et al., Liebigs Ann., 481, (1995).

### Example 5

### Synthesis of Reactive Dye V

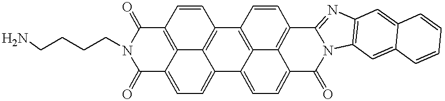

To a solution of azido protected Reactive Dye V precursor (1 mmol, 1 equiv) in 5 mL THF is added H₂O (10 mmol, 10 equiv) and tributyl phosphine (3 mmol, 3 equiv). The reaction mixture is stirred for 1 hr and then partitioned between methylene chloride and brine. The aqueous was back extracted once with methylene chloride and the combined organics were dried over anhydrous sodium sulfate. Concentration and purification by silica gel chromatography affords Reactive Dye V. The azido precursor is prepared in accordance with procedures described by Langhals, H., et. al., Liebigs Ann., 481, (1995).

### Example 6

### Derivatization of Carbohydrates with Reactive Dye V

Synthesis of High-mannose Glycoconjugate

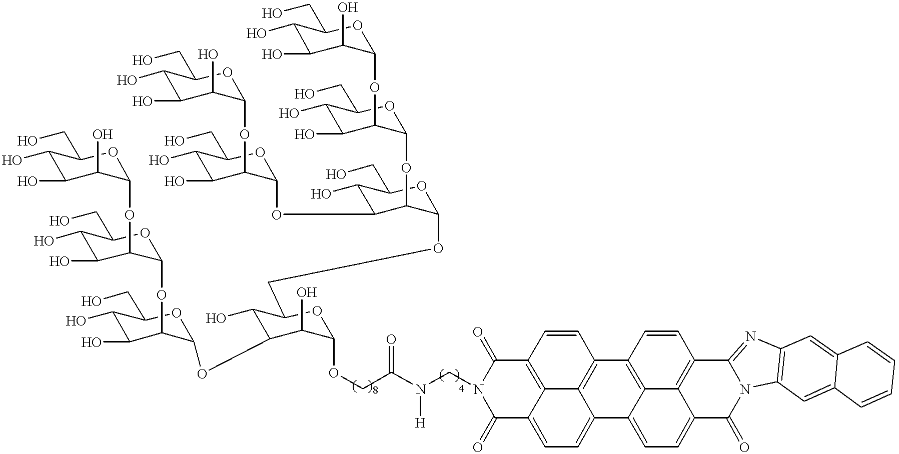

Fully acetylated high-mannoside bearing a nonanoate spacer is prepared as described by Grice P. et al., Chem. Eur. J. 3: 431 (1997). To a solution of the fully acetylated high-mannoside (1 mmol, 1 equiv) and Reactive Dye V (1 mmol, 1 equiv) in 3 mL diethyl ether is added DCC (1 mmol, 1 equiv) and a catalytic amount of DMAP. The reaction mixture is stirred for 1 hr at room temperature and filtered. Concentration and purification by silica gel chromatography affords a fully protected neoglycoconjugate. To a solution of fully acetylated mannoside-Reactive Dye V conjugate in anhydrous methanol at room temperature is added a catalytic amount of sodium methoxide. The reaction mixture is stirred at this temperature for 1 hr, quenched with Amberlite 120 plus acidic resin and filtered. Concentration and purification by reverse-phase silica gel chromatography affords the pure glycoconjugate.

### Example 7

### Incorporation of Perylene Reactive Dye I into Oligonucleotides at the 5′ Position

DNA sequences are assembled using standard phosphoramidite protocols. Oligonucleotide with the sequence 5′-GTAGGTAAG-3′ were assembled on the synthesizer. The DMT is removed from the terminal 5′ hydroxyl, and the synthesis is interrupted. The CPG containing the oligonucleotide is removed from the synthesizer, dried in vacuo and poured into a small flask. To the CPG is added 30 μmol of the perylene phosphoramidite Reactive Dye I dissolved in 0.50 mL of anhydrous dichloromethane and 0.50 mL of 0.5 M tetrazole in acetonitrile. The reaction is mixed in an argon atmosphere for 1 hour. The CPG is then filtered and rinsed with dichloromethane to remove unincorporated phosphoramidite.

The sequences are deprotected in concentrated ammonia at 55° C. overnight. The oligonucleotides are purified by denaturing polyacrylamide gel electrophoresis using 20% polyacrylamide/1% bisacrylamide/7M urea gels in 89 mM tris-borate buffer containing 2 mM Na2EDTA, pH 8.0 (1× TBE buffer). The bands are visualized by UV shadowing as well as by the naked eye, and are excised. The pure oligonucleotides are electroeluted in 0.5× TBE buffer using a Schleicher and Schuell Elutrap. All oligonucleotides are desalted before use by running the solution through a G-25 TE column according to the manufacturers specifications (Roche).

### Example 8

### Incorporation of Reactive Dye IV into Oligonucleotides at the 3′ Position

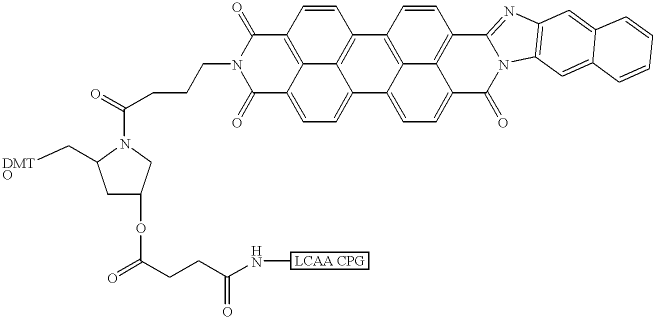

To a solution of DMT protected hydroxyprolinol (1 equiv, 1 mmol) in 3 mL dichloromethane is added Reactive Dye IV (1 equiv, 1 mmol). The reaction mixture is stirred for 1 hr and partitioned between ethyl acetate and water. The aqueous layer is back extracted with ethyl acetate and the combined organics are dried over anhydrous sodium sulfate. Concentration and purification by silica-gel chromatography affords pure hydroxyprolinol which is then coupled to succinylated aminoalkyl CPG using conditions described by Balakin, K. V. et. al., Nucleosides & Nucleotides, 18 (6&7), 1279, (1999) to give (?). This ensures the effective introduction of Reactive Dye II into the 3′ position of oligonucleotides in the course of automated nucleic acid synthesis.

### Example 9

### Labeling Antibody with Reactive Dye IV

Following procedures similar to those described in Waggoner, U.S. Pat. No. 6,048,982, to 1 mg of sheep anti-mouse-IgG antibody in 250 μL of 0.1 M sodium carbonate/bicarbonate (pH 9.2) is added 10 μl of Reactive Dye IV solution (4.42 mg/ml DMSO), giving a molar ratio of dye:protein of 10:1. The reaction mixture is stirred at 22° C. for 2 hours, and then is passed over a Sephadex G-15 desalting column (2.5 ml bed volume) that is preconditioned with phosphate buffered saline. The dye-conjugated protein obtained from the column has an apparent ratio of about 5 dyes/protein. Other reactive dyes bearing N-hydroxysuccinimide esters, isothiocyanate, or dichlorotriazine groups are attached to antibody molecules by essentially the identical procedure.

### Example 10

### Labeling Streptavidin with Reactive Dye IV

Streptavidin (5 mg/ml) and biotinylated DNA probes are purchased from Roche. A 10-mer oligonucleotide is labeled with a Biotin Chem-Link following the supplied instructions. Conjugations of streptavidin and the Reactive Dye IV are performed by dissolving Reactive Dye IV in aqueous media at a concentration of 10 mg/ml and at varied dye-to-protein molar ratios. A water-miscible organic cosolvent, such as DMSO, may be added to enhance solubility of the reactive dye compound. The reaction mixtures are incubated for 90 min at room temperature, quenched with the addition of hydroxylamine (final concentration of 0.15 M at pH 8.0) and incubated for an additional 30 min. The conjugates are purified from the unreacted dye by size-exclusion chromatography using Bio-Gel P-30 (BioRad). The biomarker-streptavidin conjugate is incubated with the biotinylated oligonucleotides by adding 300 μl buffer (100 mM MES (Sigma), 1M NaCl (Sigma), 0.05% Tween 20 (Pierce)), 24.0 μl BSA 50 mg/ml, 6.0 μl biotinylated oligo 0.5 mg/ml, 6.0 μl of biomarker-streptavidin conjugate 1 mg/ml, and 264 μl of H₂O. The reaction is incubated for 30 min, and 10 μl aliquots are removed and mixed with equal volume of a 50% sucrose loading dye. The samples are loaded on a 4% to 20% TBE, the system is loaded with 1× TBE buffer, and the gel is run at 150 volts for 1 hr. The biomarker-streptavidin conjugated biotinylated oligonucleotide is visualized as a black band running in the gel and recorded by a digital camera and a computer scanner. The location of the oligonucleotide is confirmed by staining the gel with the fluorescent nucleic acid dye SYBR Green I (Molecular Probes).

### Example 11

### Staining and Microscopic Visualization of Human Lymphocytes with Reactive Dye IV Conjugated to Sheep Antimouse IgG Antibody

Following a procedure analogous to that described in Waggoner, U.S. Pat. No. 6,048,982, freshly isolated peripheral lymphocytes are treated at zero degrees for 30 minutes with mouse anti-Beta2-microglobulin (0.25 μg/10⁶ cells). The cells are washed twice with DMEM buffer and are then treated with Reactive Dye IV-labeled sheep anti-mouse-IgG antibody (1 μg per 10⁶ cells). After a 30 minute incubation at 0° C., the excess antibody is removed and the cells are again washed twice with DMEM buffer. Aliquots of the cells are fixed on slides for analysis by microscopy by a Zeiss inverted microscope with high Numerical Aperature objectives-60× and 100×. Under the microscope the stained lymphocytes on the slide are visualized by an ICCD (intensified CCD) or EB CDD (Electron bombarded CCD) camera attached to an image digitizer and television monitor. The cells stained by this method are visible under the microscope. In a control experiment, use of the primary mouse anti-Beta2-microglobulin antibody is omitted but the staining and analysis are otherwise carried out as described above. The control sample is not visible under the microscope, indicating that Reactive Dye IV-labeled sheep anti-mouse antibody does not give significant nonspecific binding to lymphocytes.

### Example 12

### Synthesis of DNA and RNA ladders, Visible Standards of Predetermined Sizes

Oligodeoxynucleotides are synthesized on an Applied Biosystems 381A DNA Synthesizer. Protected nucleotide phosphoramidites for the four DNA bases and four RNA bases are commonly available, e.g., from Glenn Research. Oligonucleotides of various sizes are synthesized and tagged with a Reactive Dye phosphoramidite, as described in Example 7. The dye-labeled oligonucleotides are then purified by chromatographic or electrophoretic methods, and desalted by standard methods.

DNA or RNA sequences of varying sizes, each having a different color dye attached, are synthesized as described above. The labeled oligonucleotides are then mixed to give a ladder useful for tracking the sizes of unknown single stranded oligonucleotides.

In addition, the above sequences can be hybridized with their complementary sequences, combined and used as a ladder for measuring the migration of double stranded samples. DNA and RNA duplexes are hybridized by heating the complementary strands in solution for 5 min at 80° C. and then letting the mixture cool for 14-20 h. The three duplexes are mixed, and samples of 50 ng in a 5 μL volume are loaded onto 4% (29:1, acrylamide:bis) native polyacrylamide gels containing 1× TAE (90 mM Tris base (pH 8.0, 2.0 mM EDTA, 90 mM boric acid and 5% sucrose and separated by electrophoresis at room temperature in 1× TAE at approximately 25 mA (140 V) for 1 h. The three bands of different color are distinctly visible after running the gel for five minutes.

### Example 13

### Band Shift Experiment: Determination of Binding Affinity of a DNA Binding Protein for DNA Oligonucleotide Modified with a DNA Damaging Agent

DNA is labeled with Reactive Dye I, hybridized to its complementary sequence, and modified with cis-diaminedichloroplatinum(II) (cisplatin). Binding reactions are carried out in 15 μl reactions containing 20 mM Tris base, 5 mM MgCl₂, 2.5 mM CaCl₂, 0.1 mM DTT, 0.01 mM EDTA, and 50 ng of nonspecific chicken erythrocyte competitor DNA. Binding is performed at 30 min on ice. Samples are then loaded onto 4% (29:1 acrylamide:bis) native polyacrylamide gels containing 1× TAE (90 mM Tris base (pH 8.0), 2.0 mM EDTA, 90 mM boric acid) and 5% sucrose, and separated by electrophoresis at room temperature in 1× TAE at ˜25 mA (140V) for 2 h. Binding of MutS to perylene labeled 24-base pair (bp) DNA probes containing cisplatin-DNA adducts is readily observed by the retarded migration of the labeled probe through the gel. The level of binding is reflected in the fraction of shifted probe. Amounts of bound and unbound perylene labeled probe are determined by densiometric quantitative analysis. The K_{d(app)} is determined by a nonlinear least squares fitting of the binding data to the standard Hill equation.

### Example 14

### Applications of Perylene Reactive Dyes in Microarrays

DNA/RNA Micro-array

As a proof of principle, 20 bp RNA oligonucleotides are synthesized and biotinylated as described. An array is built of complementary and non-complementary sequences on a glass slide as directed on P. Brown's web page (http://cmgm.stanford.edu/pbrown/). The probes are hybridized and then stained in a buffer containing red biomarker-streptavidin conjugate as described above (Example 9). The hybridization results yield a red positive pattern of hybridization that is visualized by an ICCD (intensified CCD) or EB CDD (Electron bombarded CCD) camera attached to an image digitizer. When the identical procedure is carried out with a non-complementary sequence, no visual hybridization is detected.

Protein Microarray: Interaction of Protein G and Immunoglobulin IgG

Following the procedure described by G. MacBeath and S. Schreiber, Science, 289, 1760 (2000), glass slides are chemically derivatized and proteins are arrayed on the glass. The protein is spotted on a single aldehyde slide using a split pin arrayer constructed following directions on P. Brown's web page(http://cmgm.stanford.edu/pbrown/). Goat-anti-Mouse IgG (Pierce) is labeled with the Reactive Dye IV, as described above for Streptavidin. To probe the slides, the labeled protein is diluted into PBS, pH 7.5 supplemented with 0.1% Tween-20 (v/v) and 1% BSA (w/v), to a concentration of 0.5 mg/ml. To the slide is applied 0.55 ml of protein solution, using a PC500 CoverWell incubation chamber (Grace Biolabs). Following a 1-hour incubation at room temperature, the slides are rinsed with PBS and then washed 3 times for 3 min each with PBST (PBS supplemented with 0.1% Tween-20). The slides are rinsed twice with PBS and centrifuged at 200 g for 1 min to remove excess buffer. The slides are visualized by an ICCD (intensified CCD) or EB CDD (Electron bombarded CCD) camera attached to an image digitizer.

### Example 15

### Derivatization of Carbohydrates with Reactive dye IV

Synthesis of High-mannose Glycoconjugate (J)

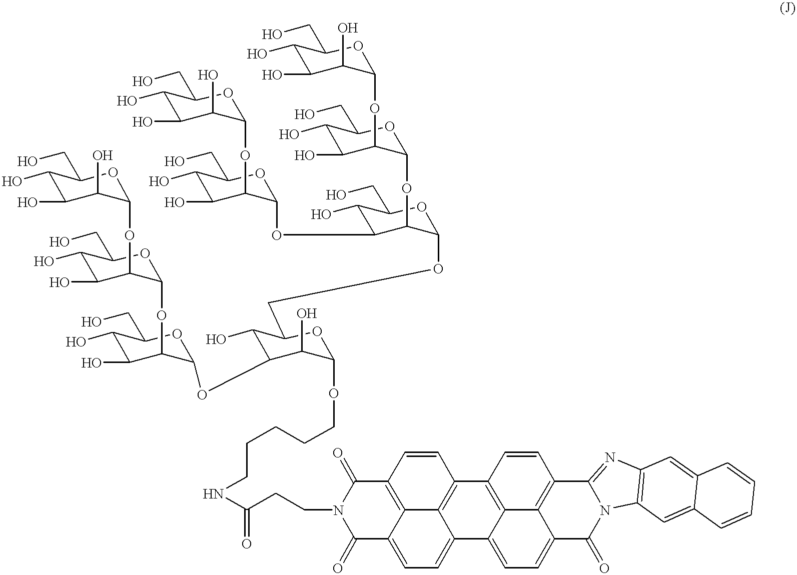

To a solution of fully acetylated 5-amino mannoside (1 mmol, 1 equiv) in 3 mL dichloromethane is added Reactive dye IV (1 mmol, 1 equiv). The reaction mixture is stirred for 1 hr and partitioned between ethyl acetate and water. The aqueous layer is back extracted with ethyl acetate ×2. The combined organics are dried over anhydrous sodium sulfate. The mixture is concentrated and purified via flash silica gel column chromatography. To a solution of fully acetylated mannoside-Reactive Dye IV conjugate in anhydrous methanol (2 mL) is added a catalytic amount of sodium methoxide (0.2 mmol, 0.2 equiv). The reaction mixture is stirred for 4 hr at room temperature, and then quenched by the addition of Amberlite 120 acidic resin. Filtration, concentration and purification by reverse-phase silica gel chromatography affords pure (J).

Carbohydrate Microarray

Following the procedure described by G. MacBeath and S. Schreiber, Science, 289, 1760 (2000), glass slides are chemically derivatized and proteins are arrayed on the glass. The protein is spotted on a single aldehyde slide using a split pin arrayer constructed following directions on P. Brown's web page(http://cmgm.stanford.edu/pbrown/). Gp120 is purchased from Pierce. To probe the slides, the carbohydrate-biomarker conjugate is diluted into PBS, pH 7.5 supplemented with 0.1% Tween-20 (v/v) and 1% BSA (w/v) to a concentration of 0.5 mg/ml. To the slide is applied 0.55 ml of protein solution, using a PC500 CoverWell incubation chamber (Grace Biolabs). Following a 1-hour incubation at room temperature, the slides are rinsed with PBS and then washed 3 times for 3 min each with PBST (PBS supplemented with 0.1% Tween-20). The slides are rinsed twice with PBS and centrifuged at 200 g for 1 min to remove excess buffer. The expected binding interactions are visualized by an ICCD (intensified CCD) or EB ICDD (Electron bombarded CCD) camera attached to an image digitizer.

While the foregoing invention has been described in some detail for purposes of clarity and understanding, it will be appreciated by one skilled in the art from a reading of this disclosure that various changes in form and detail can be made without departing from the true scope of the invention and appended claims.

What is claimed is:

1. A visually detectable biomolecule of formula B—(—L—(D)ₘ)ₙ, wherein
m and n are each an integer from one to about 5;
B is a biomolecule;
L, at each occurrence, is a spacer group comprising from one to about 10 linear atoms,
where L is attached to B by means of an ester, amide, phosphate, phosphorothioate, phosphonate, thioester, or disulfide linkage, and where the remaining linear atoms in L are selected from the group consisting of carbon, oxygen, nitrogen, and sulfur;
wherein the linear atoms in L can be optionally substituted and optionally can be included in a ring; and
D, at each occurrence, is a radical of a photostable visible dye, wherein each P has one and only one linkage to a biomolecule, provided that D is not unsubstituted perylenyl.

2. A visually detectable biomolecule of formula B—(—L—(P)ₘ)ₙ, wherein
m and n are each an integer from one to about 5;
B is a biomolecule;
L, at each occurrence, is a spacer group comprising from one to about 10 linear atoms,
where L is attached to B by means of an ester, amide, phosphate, phosphorothioate, phosphonate, thioester, or disulfide linkage, and where the remaining linear atoms in L are selected from the group consisting of carbon, oxygen, nitrogen, and sulfur;
wherein the linear atoms in L can be optionally substituted and optionally can be included in a ring; and
P, at each occurrence, is a radical of a perylene, anthracene, naphthalene, or pyrene derivative, wherein each P has one and only one linkage to a biomolecule.

3. The visually detectable biomolecule of claim 1, wherein B is selected from the group consisting of nucleic acids, carbohydrates, amino acids, and polypeptides.

4. The visually detectable biomolecule of claim 1, wherein B is a nucleic acid.

5. The visually detectable biomolecule of claim 1, wherein B is selected from the group consisting of enzymes, receptors, receptor ligands, antibodies, and glycoproteins.

6. The visually detectable biomolecule of claim 2, wherein P is a radical of a perylene derivative.

7. The visually detectable biomolecule of claim 6, wherein the perylene derivative is a perylene imide, perylene bisimide, or perylene hydrazamimide.

8. The visually detectable biomolecule of claim 7, wherein P has the formula: 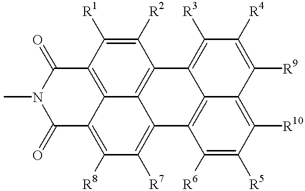
wherein
R¹, R², R³, R⁴, R⁶, R⁷, R⁸, R⁹, and R¹⁰ are radicals independently selected from the group consisting of hydrogen, halogen, cyano, hydroxy, alkoxy, amino, alkylamino, dialkylamino, heterocyclic radical, alkyl, and aryl, wherein the alkyl and aryl groups may be optionally substituted, and wherein adjacent radicals can form a carbocyclic or heterocyclic ring.

9. The visually detectable biomolecule of claim 8, wherein P has the formula: 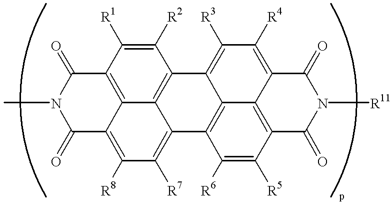
wherein p is 0, 1, or 2;
R¹, R², R³, R⁴, R⁵, R⁶, R⁷, R⁹, and R¹⁰ are radicals independently selected from the group consisting of hydrogen, halogen, cyano, hydroxy, alkoxy, amino, alkylamino, dialkylamino, heterocyclic radical, alkyl, and aryl, wherein the alkyl and aryl groups may be optionally substituted, and wherein adjacent radicals can form a carbocyclic or heterocyclic ring; and
R¹¹ is C₁-C₆ alkyl, C₆-C₁₀ aryl, or (C₆-C₁₀)ar(C₁-C₆)alkyl.

10. The visually detectable biomolecule of claim 8, wherein P has the formula: 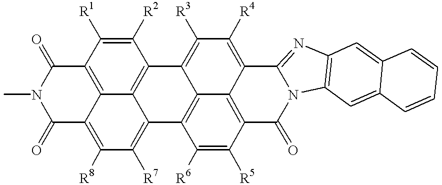
wherein
R¹, R², R³, R⁴, R^{5, R}⁶, R⁷, R⁸, R⁹, and R¹⁰ are radicals independently selected from the group consisting of hydrogen, halogen, cyano, hydroxy, alkoxy, amino, alkylamino, dialkylamino, heterocyclic radical, alkyl, and aryl, wherein the alkyl and aryl groups may be optionally substituted, and wherein adjacent radicals can form a carbocyclic or heterocyclic ring.

11. The visually detectable biomolecule of claim 7, wherein P has the formula: 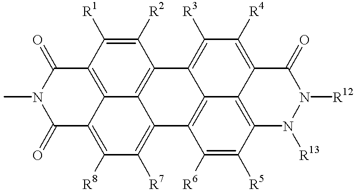
wherein
R¹, R², R³, R⁴, R⁵, R⁶, R⁷, R⁸, R⁹, and R¹⁰ are radicals independently selected from the group consisting of hydrogen, halogen, cyano, hydroxy, alkoxy, amino, alkylamino, dialkylamino, heterocyclic radical, alkyl, and aryl, wherein the alkyl and aryl groups may be optionally substituted, and wherein adjacent radicals can form a carbocyclic or heterocyclic ring; and
R¹² and R¹³ are independently C₁-C₆ alkyl, C₆-C₁₀ aryl, or (C₆-C₁₀)ar(C₁-C₆)alkyl.

12. A visually detectable biomolecule standard of formula B—(—L—(D)ₘ)ₙ, wherein
m and n are each an integer from one to about 5;
B is a biomolecule;
L is a spacer group comprising from one to about 10 linear atoms,
where L is attached to B by means of an ester, amide, phosphate, phosphorothioate, phosphonate, thioester, or disulfide linkage, and where the remaining linear atoms in L are selected from the group consisting of carbon, oxygen, nitrogen, and sulfur;
wherein the linear atoms in L can be optionally substituted and optionally can be included in a ring; and
D, at each occurrence, is a radical of a photostable visible dye, wherein each D has one and only one linkage to a biomolecule, provided that D is not unsubstituted perylenyl;
wherein the standard has a defined molecular weight.

13. A visually detectable biomolecule standard of formula B—(—L—(P)ₘ)ₙ, wherein
m and n are each an integer from one to about 5;
B is a biomolecule;
L is a spacer group comprising from one to about 10 linear atoms,
where L is attached to B by means of an ester, amide, phosphate, phosphorothioate, phosphonate, thioester, or disulfide linkage, and where the remaining linear atoms in L are selected from the group consisting of carbon, oxygen, nitrogen, and sulfur;
wherein the linear atoms in L can be optionally substituted and optionally can be included in a ring; and
P, at each occurrence, is a radical of a perylene, anthracene, naphthalene, or pyrene derivative, wherein each P has one and only one linkage to a biomolecule;
wherein the standard has a defined molecular weight.

14. A kit for determining the size of a test biomolecule, comprising a collection of two or more visually detectable biomolecule standards of formula B—(—L—(D)ₘ)ₙ, wherein
m and n are each an integer from one to about 5;
B is a biomolecule;
L is a spacer group comprising from one to about 10 linear atoms,
where L is attached to B by means of an ester, amide, phosphate, phosphorothioate, phosphonate, thioester, or disulfide linkage, and where the remaining linear atoms in L are selected from the group consisting of carbon, oxygen, nitrogen, and sulfur;
wherein the linear atoms in L can be optionally substituted and optionally can be included in a ring; and
D, at each occurrence, is a radical of a photostable visible dye, wherein each D has one and only one linkage to a biomolecule;
wherein each standard has a defined molecular weight.

15. A kit for determining the size of a test biomolecule, comprising a collection of two or more visually detectable biomolecule standards of formula B—(—L—(P)ₘ)ₙ, wherein
m and n are each an integer from one to about 5;
B is a biomolecule;
L is a spacer group comprising from one to about 10 linear atoms,
where L is attached to B by means of an ester, amide, phosphate, phosphorothioate, phosphonate, thioester, or disulfide linkage, and where the remaining linear atoms in L are selected from the group consisting of carbon, oxygen, nitrogen, and sulfur;
wherein the linear atoms in L can be optionally substituted and optionally can be included in a ring; and
P, at each occurrence, is a radical of a perylene, anthracene, naphthalene, or pyrene derivative, wherein each P has one and only one linkage to a biomolecule;
wherein each standard has a defined molecular weight.

16. A method for determining the size of a test biomolecule, comprising:
(a) subjecting the visually detectable biomolecule kit according to claim 14 or 15 to conditions under which the biomolecule standards migrate to different positions according to molecular weight, thereby producing a visual ladder of biomolecule standards;
(b) subjecting the test biomolecule to the same conditions employed in step (a); and
(c) comparing the migration of test biomolecule to the visual ladder of biomolecule standards to determine the molecular weight of the test biomolecule.

17. A method for visually detecting a biomolecule, comprising
(a) providing a biological system with a visually detectable biomolecule of formula B—(—L—(D)ₘ)ₙ, wherein
m and n are each an integer from one to about 5;
B is a biomolecule;
L is a spacer group comprising from one to about 10 linear atoms,
where L is attached to B by means of an ester, amide, phosphate, phosphorothioate, phosphonate, thioester, or disulfide linkage, and where the remaining linear atoms in L are selected from the group consisting of carbon, oxygen, nitrogen, and sulfur;
wherein the linear atoms in L can be optionally substituted and optionally can be included in a ring; and
D, at each occurrence, is a radical of a photostable visible dye, wherein each D has one and only one linkage to a biomolecule, provided that D is not unsubstituted perylenyl; and
(b) detecting the biomolecule by its visible properties.

18. A method for visually detecting a biomolecule, comprising
(a) providing a biological system with a visually detectable biomolecule of formula B—(—L—(P)ₘ)ₙ, wherein
m and n are each an integer from one to about 5;
B is a biomolecule;
L is a spacer group comprising from one to about 10 linear atoms,
where L is attached to B by means of an ester, amide, phosphate, phosphorothioate, phosphonate, thioester, or disulfide linkage, and where the remaining linear atoms in L are selected from the group consisting of carbon, oxygen, nitrogen, and sulfur;
wherein the linear atoms in L can be optionally substituted and optionally can be included in a ring; and
P, at each occurrence, is a radical of a perylene, anthracene, naphthalene, or pyrene derivative, wherein each P has one and only one linkage to a biomolecule; and
(b) detecting the biomolecule by its visible properties.

19. A method for visually detecting a biomolecule, comprising:
(a) contacting a biomolecule with a reactive dye of (D)ₙ—L—X, wherein
D is a radical of a photostable visible dye, provided that D is not unsubstituted perylenyl;
L is a spacer group comprising from one to about 10 linear atoms selected from the group consisting of carbon, oxygen, nitrogen, and sulfur;
wherein the linear atoms in L can be optionally substituted and optionally can be included in a ring; and
X is a reactive group that enables attachment to an amino, hydroxy, carboxyl, or sulfhydryl group on a biomolecule;
whereby a visually detectable biomolecule of formula B—(—L—(D)ₘ)ₙ is produced; and
(b) detecting the biomolecule by its visible properties.

20. A method for visually detecting a biomolecule, comprising:
(a) contacting a biomolecule with a reactive dye of formula (P)ₙ—L—X, wherein
P is a radical of a perylene, anthracene, naphthalene, or pyrene derivative;
L is a spacer group comprising from one to about 10 linear atoms selected from the group consisting of carbon, oxygen, nitrogen, and sulfur;
wherein the linear atoms in L can be optionally substituted and optionally can be included in a ring; and
X is a reactive group that enables attachment to an amino, hydroxy, carboxyl, or sulfhydryl group on a biomolecule;
whereby a visually detectable biomolecule of formula B—(—L—(P)ₘ)ₙ is produced; and
(b) detecting the biomolecule by its visible properties.

21. The method of claim 19, wherein B is selected from the group consisting of nucleic acids, carbohydrates, amino acids, and polypeptides.

22. The method of claim 19, wherein B is a nucleic acid.

23. The method of claim 19, wherein B is selected from the group consisting of enzymes, receptors, receptor ligands, antibodies, and glycoproteins.

24. The method of claim 19, wherein the perylene derivative is a perylene imide, perylene bisimide, or perylene hydrazamimide.

25. The method of claim 24, wherein P has the formula: 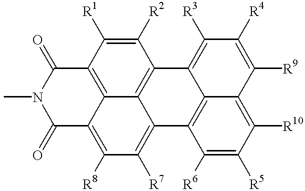
wherein
R¹, R², R³, R⁴, R⁵, R⁶, R⁷, R⁸, R⁹, and R¹⁰ are radicals independently selected from the group consisting of hydrogen, halogen, cyano, hydroxy, alkoxy, amino, alkylamino, dialkylamino, heterocyclic radical, alkyl, and aryl, wherein the alkyl and aryl groups may be optionally substituted, and wherein adjacent radicals can form a carbocyclic or heterocyclic ring.

26. The method of claim 24, wherein P has the formula: 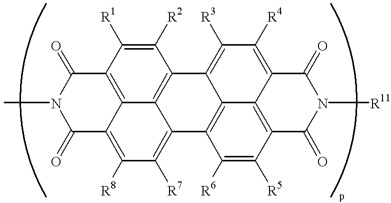
wherein p is 0, 1, or 2; and R¹¹ is C₁-C₆ alkyl, C₆-C₁₀ aryl, or (C₆-C₁₀)ar(C₁-C₆)alkyl.

27. The method of claim 24, wherein P has the formula:

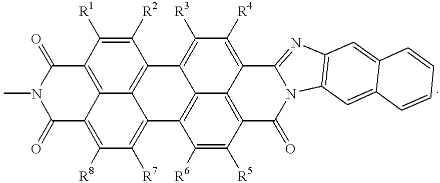

28. A reactive dye of formula(D)ₙ—L—X, wherein
n is an integer from 1 to about 5;
D is a radical of a photostable visible dye, provided that D is not unsubstituted perylenyl;
L is a spacer group comprising from one to about 10 linear atoms selected from the group consisting of carbon, oxygen, nitrogen, and sulfur;
wherein the linear atoms in L can be optionally substituted and optionally can be included in a ring; and
X is 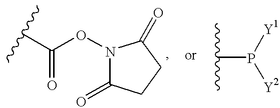
wherein Y¹ and Y² are independently dialkylamino, N-heterocyclic radical, or OZ, where Z is a protecting group.

29. A reactive dye of formula(P)ₙ—L—X, wherein
n is an integer from 1 to about 5;
P is a radical of a perylene, anthracene, naphthalene, or pyrene derivative;
L is a spacer group comprising from one to about 10 linear atoms selected from the group consisting of carbon, oxygen, nitrogen, and sulfur;
wherein the linear atoms in L can be optionally substituted and optionally can be included in a ring; and
X is 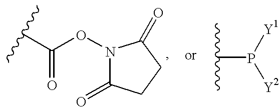
wherein Y¹ and Y² are independently dialkylamino, N-heterocyclic radical, or OZ, where Z is a protecting group.

30. A reactive dye of formula (P)ₙ—L—X, wherein
n is an integer from 1 to about 5;
P is a radical of a perylene derivative having a formula selected from the group consisting of: 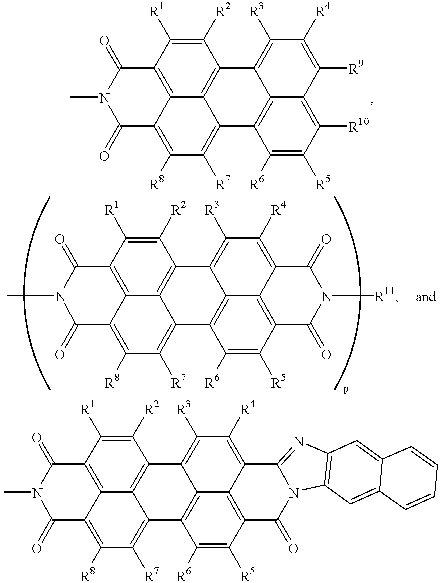
wherein
R¹, R², R³, R⁴, R⁵, R⁶, R⁷, R⁸, R⁹, and R¹⁰ are radicals independently selected from the group consisting of hydrogen, halogen, cyano, hydroxy, alkoxy, amino, alkylamino, dialkylamino, heterocyclic radical, alkyl, and aryl, wherein the alkyl and aryl groups may be optionally substituted, and wherein adjacent radicals can form a carbocyclic or heterocyclic ring;
p is 0, 1, or 2;
R¹¹ is C₁-C₆ alkyl, C₆-C₁₀ aryl, or (C₆-C₁₀)ar(C₁-C₆)alkyl;
L is a spacer group comprising from one to about 10 linear atoms selected from the group consisting of carbon, oxygen, nitrogen, and sulfur;
wherein the linear atoms in L can be optionally substituted and optionally can be included in a ring; and
X is a reactive group that enables attachment to an amino, hydroxy, carboxyl, or sulfhydryl group on a biomolecule.

31. The reactive dye of claim 30, wherein X has the formula: 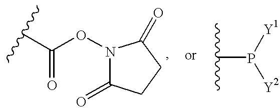
wherein Y¹ and Y² are independently dialkylamino, N-heterocyclic radical, or OZ, where Z is a protecting group.

32. The reactive dye of claim 29 or 31, wherein Z is alkyl, allyl, aryl, or cyanoalkyl.

33. The reactive dye of claim 29 or 31, wherein Y¹ is cyanoethyl and Y² is diisopropylamino.
